(19) United States
(12) Patent Application Publication
(10) Pub. No.: US 2024/0354469 A1
(43) Pub. Date: Oct. 24, 2024

(54) METHOD FOR OPTIMIZING LAYER-STACKING SEQUENCE OF COMPOSITE MATERIAL PRESSURE VESSEL

(71) Applicant: Taiyuan University of Technology, Taiyuan City (CN)

(72) Inventors: Jianguo LIANG, Taiyuan City (CN); Qingxue HUANG, Taiyuan City (CN); Zemin NING, Taiyuan City (CN); Jianglin LIU, Taiyuan City (CN); Yinhui LI, Taiyuan City (CN); Yanchun ZHU, Taiyuan City (CN); Zhaotun JIA, Taiyuan City (CN); Chunxiang MIAO, Taiyuan City (CN); Xiaodong ZHAO, Taiyuan City (CN); Runtian ZHAO, Taiyuan City (CN)

(21) Appl. No.: 18/543,391

(22) Filed: Dec. 18, 2023

(30) Foreign Application Priority Data
Apr. 20, 2023 (CN) 202310426963.7

(51) Int. Cl. G06F 30/23; G06F 111/10; G06F 113/26

(52) U.S. Cl. CPC G06F 30/23; G06F 2111/10; G06F 2113/26

(57) ABSTRACT

A method for optimizing a layer-stacking sequence of a composite material pressure vessel. includes determining design variables of a to-be-optimized composite material pressure vessel based on design objectives thereof, the design objectives including a liner size and a working pressure of the composite material pressure vessel, and the design variables including filament winding angles, winding layer thicknesses, and winding layer quantities; establishing a finite element model based on layer-stacking information and boundary conditions of the to-be-optimized composite material pressure vessel, the layer-stacking information being the design variables; modeling the liner of the pressure vessel by solid elements, and modeling composite material layers of the pressure vessel by continuum shell elements. The method includes obtaining required optimization objectives based on the established finite element model; and establishing an optimization model based on the optimization objectives, and obtaining, through iterative optimization, an optimal layer-stacking sequence of the composite material pressure vessel.

### FIELD OF TECHNOLOGY

The present disclosure falls within the field of composite material pressure vessel simulation, and relates to a method for optimizing a layer-stacking sequence of a composite material pressure vessel.

### BACKGROUND

Composite material pressure vessels have advantages of low specific gravity, good fatigue resistance, and high safety, and play an important role in aircraft, vehicle-mounted pressure vessels, petrochemical industry, and other fields. Compared with conventional metallic pressure vessels, the composite material multi-layer structure adds more room for imagination to the design of pressure vessels. According to different design requirements and application conditions and with different layer designs, component material matching, and interface control, the advantages of composite materials can be maximized to meet application needs in different occasions. In terms of structural designs of composite material pressure vessels, most manufacturers and researchers may determine a spirally winding layer quantity and a circumferentially winding layer quantity based on the netting theory and adopt empirical design for the layer-stacking sequence after determining the spirally winding layer quantity and the circumferentially winding layer quantity. Optimization designs of composite material layer structures are widely used, but concentrating on optimization designs of winding angles and winding thicknesses. Composite material pressure vessels generally have many winding layers at various angles. Meanwhile, the constraints of the winding process are taken into account, and the design process is complex. The layer-stacking sequence design can improve the strength of composite material pressure vessels, thus having great significance.

### SUMMARY

In order to solve the problems above, the present disclosure provides a method for optimizing a layer-stacking sequence of a composite material pressure vessel.

The present disclosure adopts the following technical solution: a method for optimizing a layer-stacking sequence of a composite material pressure vessel, including:
S1: determining design variables of a to-be-optimized composite material pressure vessel based on design objectives thereof, the design objectives including a liner size and a working pressure of the composite material pressure vessel, and the design variables including filament winding angles, winding layer thicknesses, and winding layer quantities;
S2: establishing a finite element model based on layer-stacking information and boundary conditions of the to-be-optimized composite material pressure vessel, the layer-stacking information being the design variables in step S1; modeling the liner of the pressure vessel by solid elements, and modeling composite material layers of the pressure vessel by continuum shell elements;
S3: obtaining required optimization objectives based on the established finite element model; and
S4: establishing an optimization model based on the optimization objectives, and obtaining, through iterative optimization, an optimal layer-stacking sequence of the composite material pressure vessel in a case that the design objectives and the design variables are determined.

In step S1, the pressure vessel includes a body section and a cap section in structure, composite material layers of the body section include spirally winding layers and circumferentially winding layers, and composite material layers of the cap section include spirally winding layers; and a winding layer quantity of the body section is a winding layer quantity of the cap section;

the filament winding angles are determined by reaming winding;

the filament winding angles of the body section are calculated by the following formula:

$${α}_{1} = {sin}^{- 1} \frac{{r}_{0} + {r}_{i}}{R} ,$$

in the formula, r₀ is a polar hole radius, rᵢ is a reaming radius, R is a liner radius, and αᵢ is a filament winding angle of the body section corresponding to a distinct reaming radius;

the filament winding angles of the cap section are calculated by the following formula:

$${α}_{i} ( r ) = {sin}^{- 1} \frac{{r}_{0} + {r}_{i}}{r} ,$$

in the formula, r is a parallel circle radius at each position of the cap, and αᵢ (r) is a filament winding angle at each position of the cap corresponding to a distinct reaming radius;

the winding layer thicknesses of the body section are calculated by the following formula:

$$\{ \begin{matrix}{t}_{f α} = \frac{{RP}_{b}}{2 K {σ}_{max} {cos}^{2} α} \\ {t}_{f θ} = \frac{{RP}_{b}}{2 {σ}_{max}} ( 2 - {tan}^{2} α )\end{matrix}$$

in the formula, t_{fα}. is a spirally winding layer thickness of the body section, t_{fθ} is a circumferentially winding layer thickness of the body section, P_{b} is a design burst pressure of the body section, K is a filament strength utilization coefficient, σₘₐₓ is a filament tensile strength, and α is the filament winding angle of the body section;

the winding layer thicknesses of the cap section are calculated by the following formula:

$${t}_{f} ( r ) = {\sum }_{i} \sqrt{\frac{{R}^{2} - {( {r}_{0} + {r}_{i} )}^{2}}{{r}^{2} - {( {r}_{0} + {r}_{i} )}^{2}}} {t}_{f α i}$$

in the formula, t_{f} (r) is a winding layer thickness at a distinct parallel circle radius of the cap section, R is the liner radius, r₀ is the polar hole radius, rᵢ is the reaming radius, r is the parallel circle radius at each position of the cap, and t_{fαi} is a winding layer thickness at a distinct filament winding angle of the body section; and

the winding layer quantities are calculated by the following formula:

$$\{ \begin{matrix}M = \frac{{t}_{f α}}{t} \\ N = \frac{{t}_{f θ}}{t}\end{matrix}$$

in the formula, M is a spirally winding layer quantity, N is a circumferentially winding layer quantity, and t is a thickness of a single filament layer.
a. In step S2, the finite element model of the composite material pressure vessel refers to establishing a static model and a thermodynamic model separately using the same layer-stacking information;

boundary conditions for the static model include: a predefined field including four analysis steps, which are self-tightening, unloading, working, and bursting in sequence; and

boundary conditions for the thermodynamic model include: a predefined field including two analysis steps, which are heat preservation and temperature reduction in sequence.

In step S2, the cap section is divided into a plurality of circular rings, and winding layer angles of the cap section are assigned based on different winding layer angles corresponding to different parallel circle radii; a discrete coordinate system is used for setting winding angles for both the cap section and the body section; and a symmetrical winding layer-stacking method is used for layer-stacking.

In step S3, the optimization objectives include a minimized maximum filament-direction stress under a minimum burst pressure and a minimized maximum thermal curing deformation.

Step S4 specifically includes:

S41: determining an initial population, an iteration quantity, and a fitness function of a layer sequence, the fitness function using a maximum filament-direction stress x under a minimum burst pressure and a maximum thermal curing deformation y of each of individuals in the population as variables, the fitness function being denoted as

$$F ( x , y ) = \frac{1}{xy} ,$$

and a greater fitness function value indicating that an individual that is more excellent and has a greater probability to be selected in a selection operation;

S42: adopting integer coding for the layer sequence, with the layers occurring in pairs at positive and negative angles, each pair of layers being coded with a distinct integer so as to randomly generate an integer code sequence population; defining the layer sequence of the composite material pressure vessel by the integer code sequence: [a₁, a₂, . . . , a_{(n+m)/2}], the integer code sequence having

$$\frac{n + m}{2}$$

integers, with

$$\frac{n}{2}$$

integers representing circumferentially winding layers and

$$\frac{m}{2}$$

integers representing spirally winding layers, n being a circumferentially winding layer quantity and m being a spirally winding layer quantity; generating a layer sequence population based on layer-stacking angles represented by the integer codes, with each individual in the population representing a possible layer-stacking mode of the composite material pressure vessel;

S43: performing calculation on the layer sequence population to obtain the maximum filament-direction stress under a minimum burst pressure and the maximum thermal curing deformation corresponding to each individual in the population, and calculating the fitness function value corresponding to each individual in the population;

S44: generating a new integer code sequence population through a selection operation, a crossover operation, and a mutation operation, decoding the integer code sequence to generate a layer sequence population, and calculating corresponding fitness function values; and

S45: determining whether the iteration quantity is reached, and in a case that the iteration quantity is reached, outputting an optimal individual, and in a case that the iteration quantity is not reached, performing step S44.
a. The generating a new integer code sequence population through a selection operation, a crossover operation, and a mutation operation in step S44 specifically includes:

in the selection operation: adopting a random ergodic sampling selection, the principle of the random ergodic sampling selection being as follows: partitioning a wheel based on proportions of individual fitness function values in an accumulated population fitness function value, uniformly arranging pointers in a quantity which is a quantity of individuals to be selected, rotating the wheel, and completing selection based on partitions pointed by the pointers;

in the crossover operation: adopting order crossover which specifically includes: firstly performing adjacent pairing on a population selected by the selection operation, and determining whether the crossover operation occurs based on a crossover probability, and in a case that the crossover operation occurs, randomly selecting two crossover points from chromosomes of two parents P1 and P2; extracting genes between the two points and placing the genes at the same positions of chromosomes of two offsprings O1 and O2; for the offspring O1, arranging the chromosome of the parent P2 in order and deleting genes already existing in the offspring O1 so as to obtain chromosome sequences inherited from the parent P2 of the offspring O1, and successively inserting the chromosome sequences inherited from the parent P2 into vacant positions of the chromosome of the offspring O1 to obtain the offspring O1; obtaining the offspring O2 in the same way; and completing the crossover operation;

in the mutation operation: determining whether the mutation operation occurs based on a mutation probability, and in a case that the mutation operation occurs, randomly selecting two gene codes of individuals for exchange and completing the mutation operation; and

generating a new integer code sequence population through the three operations sequentially, and recombining a temporary population generated by selection, crossover, and mutation operations on a population of the n^{th} generation with part of excellent individuals of the population of the n^{th} generation so as to generate a population of the (n+1)^{th} generation.

Compared with the prior art, the present disclosure has the following advantageous effects.

The present disclosure solves a long-standing problem of a manual optimization by a manual trial-and-error method due to a lack of a mature fitness function system in the field of layer optimization of composite material pressure vessels based on Abaqus simulation, and significantly reduces the time for numerical simulation. The present disclosure provides a method for optimizing a layer-stacking sequence of a composite material pressure vessel which builds a layer-stacking sequence optimization design platform by writing a data interaction program of Abaqus and Python and constructing a genetic algorithm for optimizing a layer-stacking sequence of a composite material pressure vessel. The objective function is to minimize the maximum filament-direction stress under a minimum burst pressure of the composite material pressure vessel and to minimize the maximum thermal curing deformation of the composite material pressure vessel. The layer-stacking sequence of the composite material pressure vessel is optimized with filament winding angles, winding layer thicknesses, and winding layer quantities as constraint conditions. Conventional optimization methods are achieved through empirical design and classified discussions, requiring that finite element models be submitted for calculations and modifications one by one. However, the present disclosure can significantly reduce the time for numerical simulation of the composite material pressure vessel.

The present disclosure improves the design accuracy. The optimizing a layer-stacking sequence of a composite material pressure vessel is a combinatorial optimization problem. The genetic algorithm has a good global search capability, automatically acquires and accumulates knowledge of the search space during the search process, and adaptively controls the search process. Compared with the conventional optimization methods based on empirical design and classified discussions, the method for optimizing a layer-stacking sequence of a composite material pressure vessel using the genetic algorithm is more likely to obtain an optimal solution and improve the design accuracy of the composite material pressure vessel.

### BRIEF DESCRIPTION OF DRAWINGS

FIG. 1 is a flowchart of a method for optimizing a layer-stacking sequence of a composite material pressure vessel of the present disclosure;

FIG. 2 is a structural diagram of an example liner of a method for optimizing a layer-stacking sequence of a composite material pressure vessel of the present disclosure;

FIG. 3 is a schematic diagram of individual pairing of a method for optimizing a layer-stacking sequence of a composite material pressure vessel of the present disclosure;

FIG. 4 is a schematic diagram of a crossover operation of a method for optimizing a layer-stacking sequence of a composite material pressure vessel of the present disclosure;

FIG. 5 is a schematic diagram of a mutation operation of a method for optimizing a layer-stacking sequence of a composite material pressure vessel of the present disclosure;

FIG. 6 is a schematic diagram of the interaction of the optimization program with the analysis software of a method for optimizing a layer-stacking sequence of a composite material pressure vessel of the present disclosure;

FIG. 7 is a variation graph of a fitness function value during an example optimization process of a method for optimizing a layer-stacking sequence of a composite material pressure vessel of the present disclosure;

FIG. 8 is a variation graph of a maximum filament-direction stress under a minimum burst pressure during an example optimization process of a method for optimizing a layer-stacking sequence of a composite material pressure vessel of the present disclosure; and

FIG. 9 is a variation graph of a maximum thermal curing deformation during an example optimization process of a method for optimizing a layer-stacking sequence of a composite material pressure vessel of the present disclosure.

### DETAILED DESCRIPTION OF ILLUSTRATIVE EMBODIMENTS

The implementations of the present disclosure are described in further detail below with reference to the accompanying drawings and specific embodiments. The following embodiments or drawings serve to illustrate the present disclosure, without limiting the scope thereof.

As shown in FIG. 1, the present disclosure provides a method for optimizing a layer-stacking sequence of a composite material pressure vessel, specifically including the following steps.

At S1, design variables of a to-be-optimized composite material pressure vessel are determined based on design objectives thereof, the design objectives include a liner size and a working pressure of the composite material pressure vessel, and the design variables include filament winding angles, winding layer thicknesses, and winding layer quantities.

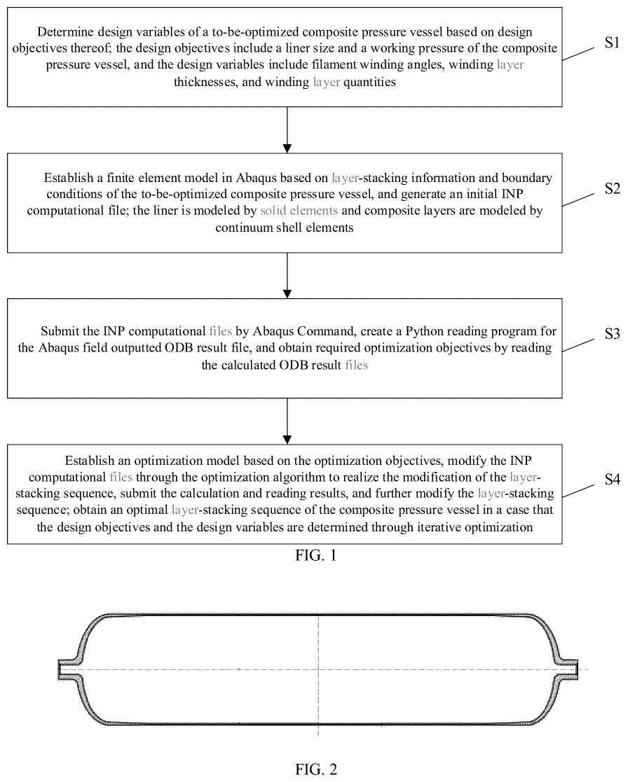

In this embodiment, the liner size of the composite material pressure vessel is shown in FIG. 2, the working pressure is 35 MPa, the filament winding angles are determined by reaming winding, and the filament winding angles of the body section are calculated by the following formula:

$${α}_{i} = {sin}^{- 1} \frac{{r}_{0} + {r}_{i}}{R} . \qquad a$$

In the formula, r₀ is a polar hole radius, rᵢ is a reaming radius, R is a liner radius, and αᵢ is a filament winding angle of the body section corresponding to a distinct reaming radius; r₀=17.5 mm, R=100 mm, and a bandwidth of a winding tow is 12 mm. The reaming winding is performed within two bandwidths, with the reaming radius r; being 0 mm, 6 mm, 12 mm, 18 mm, and 24 mm, successively, and the corresponding filament winding angles &; are calculated as 11°, 14°, 18°, 21°, and 25°, successively.

The filament winding angles of the cap section are calculated by the following formula:

$${α}_{i} ( r ) = {sin}^{- 1} \frac{{r}_{0} + {r}_{i}}{r} . \qquad a$$

In the formula, r₀ is the polar hole radius, rᵢ is the reaming radius, r is a parallel circle radius at each position of the cap, and αᵢ (r) is a filament winding angle at each position of the cap corresponding to a distinct reaming radius; r₀=17.5 mm, and the bandwidth of the winding tow is 12 mm. The reaming winding is performed within two bandwidths, with the reaming radius rᵢ being 0 mm, 6 mm, 12 mm, 18 mm, and 24 mm, successively, and the spiral winding angles corresponding to different parallel circle radii are calculated and shown in the table below,

| r/mm | 92.00 | 83.54 | 73.52 | 62.50 | 56.66 | 50.56 | 44.61 | 41.50 | 38.34 |
|---|---|---|---|---|---|---|---|---|---|
| α₀/° | 11° | 13° | 14° | 17° | 18° | 21° | 24° | 25° | 28° |
| α₁/° | 15° | 17° | 19° | 23° | 25° | 28° | 32° | 35° | 38° |
| α₂/° | 19° | 21° | 24° | 29° | 32° | 36° | 42° | 46° | 51° |
| α₃/° | 23° | 26° | 29° | 35° | 39° | 45° | 53° | 59° | 68° |
| α₄/° | 27° | 30° | 35° | 42° | 48° | 56° | 69° | 90° | |

| r/mm | 35.50 | 32.14 | 29.50 | 25.89 | 23.50 | 19.68 | 17.50 |
|---|---|---|---|---|---|---|---|
| α₀/° | 30° | 33° | 37° | 43° | 49° | 62° | 90° |
| α₁/° | 42° | 47° | 53° | 66° | 90° | | |
| α₂/° | 57° | 57° | 90° | | | | |
| α₃/° | 90° | | | | | | |
| α₄/° | | | | | | | |

The winding layer thicknesses of the body section are calculated by the following formula:

$$\{ \begin{matrix}{t}_{f α} = \frac{{RP}_{b}}{2 K {σ}_{max} {cos}^{2} α} \\ {t}_{f θ} = \frac{{RP}_{b}}{2 {σ}_{max}} ( 2 - {tan}^{2} α )\end{matrix} .$$

In the formula, t_{fα}. is a spirally winding layer thickness of the body section, t_{fθ} is a circumferentially winding layer thickness of the body section, P_{b} is a design burst pressure of the body section, K is a filament strength utilization coefficient, σₘₐₓ is a filament tensile strength, and α is the filament winding angle of the body section; R=100 mm, P_{b}=87.5 MPa, K=0.8, σₘₐₓ=2200 MPa, α=11°, and then t_{fα}=2.58 mm and t_{fθ}=3.90 mm are calculated.

The winding layer thicknesses of the cap section are calculated by the following formula:

$${t}_{f} ( r ) = {\sum }_{i} \sqrt{\frac{{R}^{2} - {( {r}_{0} + {r}_{i} )}^{2}}{{r}^{2} - {( {r}_{0} + {r}_{i} )}^{2}}} {t}_{f α i}$$

In the formula, t_{f} (r) is a winding layer thickness at a distinct parallel circle radius of the cap section, R is the liner radius, r₀ is the polar hole radius, rᵢ is the reaming radius, r is the parallel circle radius at each position of the cap, and t_{fαi} is a winding layer thickness at a distinct spiral winding angle of the body section; R=100 mm, r₀=17.5 mm, rᵢ is 0 mm, 6 mm, 12 mm, 18 mm, and 24 mm, successively, and t_{fαi} is 0.8 mm, 0.8 mm, 0.8 mm, 0.4 mm, and 0.4 mm, successively.

| r/mm | 92.00 | 83.54 | 70.62 | 62.50 | [symbol] | 50.56 | 44.61 | 41.50 | 38.34 |
|---|---|---|---|---|---|---|---|---|---|
| t [symbol] | 8.51 | 3.91 | 4.54 | 5.55 | [symbol] | 7.56 | [symbol] | 5.72 | 10.37 |

| r/mm | [symbol] | [symbol] | [symbol] | 25.89 | 22.50 | 19.88 |
|---|---|---|---|---|---|---|
| t [symbol] | 9.34 | 12.45 | [symbol] | 11.29 | 5.02 | 8.35 |

[symbol]

indicates data missing or illegible when filed

The winding layer quantities are calculated by the following formula:

$$\{ \begin{matrix}M = \frac{{t}_{f α}}{t} \\ N = \frac{{t}_{f θ}}{t}\end{matrix}$$

In the formula, M is a spirally winding layer quantity, N is a circumferentially winding layer quantity, t_{fα} is the spirally winding layer thickness of the body section, t_{fθ} is the circumferentially winding layer thickness of the body section, and t is a thickness of a single winding layer; t=0.2 mm, and then M=12.9 and N=19.5 are calculated. A symmetrical winding layer-stacking method is used for layer-stacking. The axial load bearing capacity decreases and the circumferential load bearing capacity increases with the increase of the spiral winding angle. Therefore, in order to ensure load bearing uniformity, the spirally winding layer quantity is increased and the circumferentially winding layer quantity is decreased, M=16, and N=18. The layer quantities corresponding to the spiral winding angles 11°, 14°, 18°, 21°, and 25° are 4, 4, 4, 2, and 2, separately. The circumferential winding angle is 88°, the layer quantity is 18, and each filament winding angle occurs in a pair of positive and negative angles.

At S2, a finite element model is established in Abaqus based on layer-stacking information and boundary conditions of the to-be-optimized composite material pressure vessel, and initial INP computational files are generated; the liner is modeled by solid elements and composite material layers are modeled by continuum shell elements.

It is a conventional technique in the art to establish a finite element model based on layer-stacking information and boundary conditions. However, there are various choices of boundary conditions, which may determine the quality of the model. It is a feature of the finite element model described in the present disclosure that the liner is modeled by the solid elements and the composite material layers are modeled by continuum shell elements. The INP files are those supported by Abaqus command line for computation, and Abaqus is the only commercial software that can generate the INP files. The layer-stacking information, i.e., the design variables in step S1, includes filament winding angles, winding layer thicknesses, and winding layer quantities.

In this embodiment, AL6061 aluminum alloy is selected as the liner material, and T700S carbon fiber composite material is selected as the layer material. A static model and a thermodynamic model are separately established using the same layer-stacking information, and the INP computational files are generated. The boundary conditions for the static model include: a predefined field including four analysis steps, which are self-tightening, unloading, working, and bursting in sequence, and the internal pressure loads applied to the four analysis steps of this embodiment are 52.5 MPa, 0 MPa, 35 MPa, and 87.5 MPa in sequence. The boundary conditions for the thermodynamic model include: a predefined field including two analysis steps, which are heat preservation and temperature reduction in sequence, and the ambient temperatures applied to the two analysis steps of this embodiment are 177° C. and 25° C. in sequence. The cap section of the composite material layers is divided into a plurality of circular rings, and winding layer angles of the cap section are assigned based on different winding layer angles corresponding to different parallel circle radii; a discrete coordinate system is used for setting winding angles for both the cap section and the body section; and a symmetrical winding layer-stacking method is used for layer-stacking.

At S3, the INP computational files are submitted by Abaqus Command, and a Python reading program is created for the Abaqus field outputted ODB result files, and the required performance indexes are obtained by reading the calculated ODB result files, so as to determine the optimization objectives.

The performance indexes include a maximum filament-direction stress in the analysis step of working, a maximum filament-direction stress in the analysis step of bursting, and a maximum thermal curing deformation. The optimization objectives in this embodiment are to minimize the maximum filament-direction stress under a minimum burst pressure and to minimize the maximum thermal curing deformation.

At S4, an optimization model is established based on the optimization objectives, the INP computational files are modified through the optimization algorithm to realize the modification of the layer-stacking sequence, the calculation and reading results are submitted, and the layer-stacking sequence is further modified; an optimal layer-stacking sequence of the composite material pressure vessel in a case that the design objectives and the design variables are determined is obtained through iterative optimization.

The optimization model of this embodiment has cooperative optimizations of the maximum filament-direction stress under a minimum burst pressure of the composite material pressure vessel and the maximum thermal curing deformation as the optimization objectives, calculates the optimization model by using the genetic algorithm with the filament winding angles, winding layer thicknesses, and winding layer quantities as the constraint conditions, and the implementation method is as follows.

At S41, an initial population, an iteration quantity, and a fitness function of a layer sequence are determined, the fitness function uses a maximum filament-direction stress x under a minimum burst pressure and a maximum thermal curing deformation y of each of individuals in the population as variables, the fitness function is denoted as F (x,y)=1/xy, and a greater fitness function value indicates that an individual is more excellent and has a greater probability to be selected in a selection operation.

In this embodiment, the initial population of the layer sequence is 40 and the iteration quantity is 40.

At S42, integer coding is adopted for the layer sequence, with the layers occurring in pairs at positive and negative angles, each pair of layers are coded with a distinct integer so as to randomly generate an integer code sequence population; the Abaqus INP input files are modified based on layer-stacking angles represented by the integer codes so as to generate a layer sequence population, with each individual in the population representing a possible layer-stacking mode of the composite material pressure vessel.

A program is established for modifying the content of a specified line of the INP input files, and the INP modification program can realize corresponding modifications on the winding angles in the INP input files based on different integers read in. In this embodiment, the circumferentially winding layers at 88° at the 18th layer have codes of 1, 2, 3, 4, 5, 6, 7, 8, and 9; the spirally winding layers at 11° at the 4th layer have codes of 10 and 11; the spirally winding layers at 14° at the 4th layer have codes of 12 and 13; the spirally winding layers at 18° at the 4th layer have codes of 14 and 15; the spirally winding layer at 21° at the 2nd layer has a code of 16; and the spirally winding layer at 25° at the 2nd layer has a code of 17. The integer code sequence population is randomly generated, and the INP modification program is invoked to modify the INP input files in batches based on the layer-stacking angles represented by the integer codes.

At S43, Abaqus Command batch submission and calculation is performed on the layer sequence population, and the ODB result files are read by using the Python reading program to obtain the maximum filament-direction stress under a minimum burst pressure and the maximum thermal curing deformation corresponding to each individual in the population, and the fitness function value corresponding to each individual in the population is calculated.

At S44, a new integer code sequence population is generated through a selection operation, a crossover operation, and a mutation operation, the integer code sequence is decoded to generate a layer sequence population, and corresponding fitness function values are calculated, which specifically includes the following steps.

In the selection operation, a random ergodic sampling selection is adopted, the principle of the random ergodic sampling selection are as follows: partitioning a wheel based on proportions of individual fitness function values in an accumulated population fitness function value, uniformly arranging pointers in a quantity which is a quantity of individuals to be selected, rotating the wheel, and completing selection based on partitions pointed by the pointers. In this embodiment, when a selection probability is 0.9, the quantity of individuals to be selected is 36.

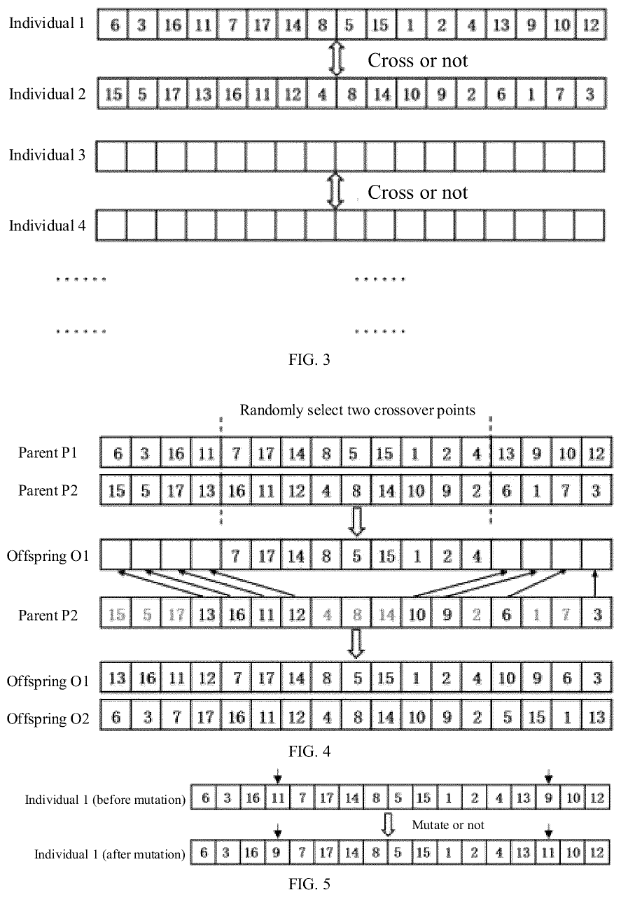

In the crossover operation, order crossover is adopted, which specifically includes: firstly performing adjacent pairing on a population selected by the selection operation, as shown in FIG. 3, and determining whether the crossover operation occurs based on a crossover probability, and in a case that the crossover operation occurs, randomly selecting two crossover points from chromosomes of two parents P1 and P2; extracting genes between the two points and placing the genes at the same positions of chromosomes of two offsprings O1 and O2; for the offspring O1, arranging the chromosome of the parent P2 in order and deleting genes already existing in the offspring O1 so as to obtain chromosome sequences inherited from the parent P2 of the offspring O1, and successively inserting the chromosome sequences inherited from the parent P2 into vacant positions of the chromosome of the offspring O1 to obtain the offspring O1; obtaining the offspring O2 in the same way; and completing the crossover operation, as shown in FIG. 4. The crossover probability is 0.95 in this embodiment.

In the mutation operation, as shown in FIG. 5, it is determined whether the mutation operation occurs based on a mutation probability, and in a case that the mutation operation occurs, two gene codes of individuals are randomly selected for exchange and the mutation operation is completed. A new integer code sequence population is generated through the three operations above in turn. A temporary population generated by selection, crossover, and mutation operations on a population of the n^{th} generation is recombined with part of excellent individuals of the population of the n^{th} generation so as to generate a population of the (n+1)^{th} generation. The mutation probability is 0.05 in this embodiment.

At S45, it is determined whether the iteration quantity is reached, and in a case that the iteration quantity is reached, an optimal individual is outputted, and in a case that the iteration quantity is not reached, step S44 is performed. The iteration quantity is 40 in this embodiment.

An Abaqus-Python-Matlab co-simulation optimization platform is built. Firstly, the finite element model is established in Abaqus, and the initial INP computational files are generated. Secondly, the INP computational files are submitted by Abaqus Command. Thirdly, the Python reading program is created for the Abaqus field outputted ODB result files, and the data required for optimization is obtained by reading the calculated ODB result files. Finally, the INP computational files are modified through Matlab, and submitted for calculation. Loop iterations are performed until the optimization condition is reached.

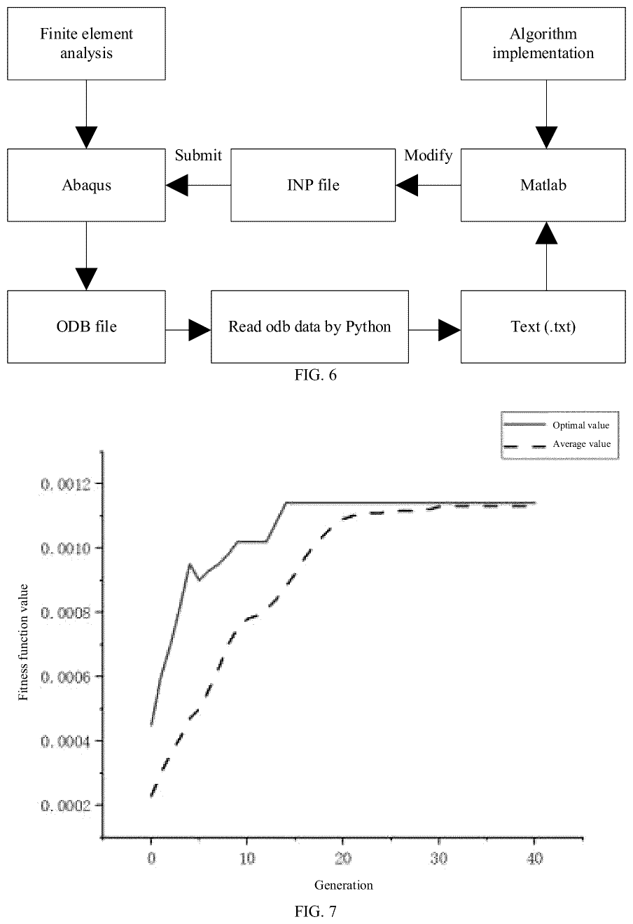

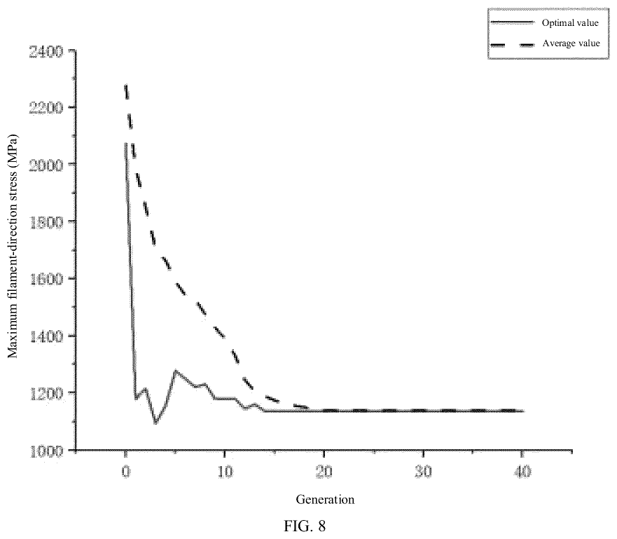

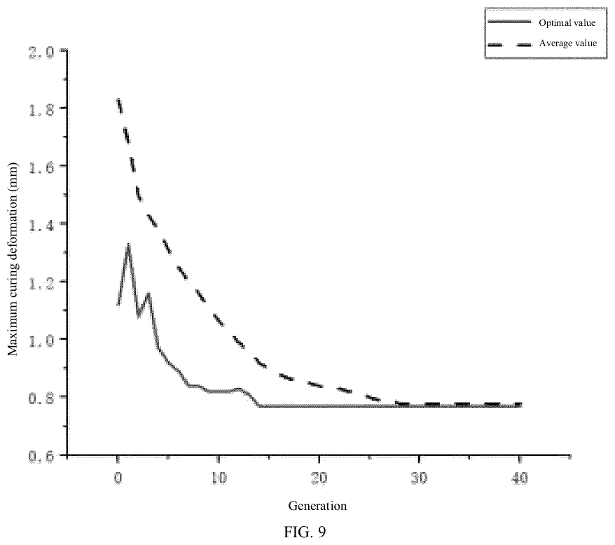

During the iteration, there is information interaction between the optimization program and the Abaqus software, as shown in FIG. 6. The variation curves of an optimal individual fitness function value and an average population fitness function value of each generation versus the iteration quantity in this embodiment are shown in FIG. 7. The variation curves of an optimal individual maximum filament-direction stress under a minimum burst pressure and an average population maximum filament-direction stress under a minimum burst pressure of each generation versus the iteration quantity in this embodiment are shown in FIG. 8. The variation curves of an optimal individual maximum thermal curing deformation and an average population maximum thermal curing deformation of each generation versus the iteration quantity in this embodiment are shown in FIG. 9.

In this embodiment, the optimal individual fitness function value of a population of the first generation is 0.43×10⁻³, and the layer-stacking sequence scheme represented by the optimal individual is [±88°₃/±18°₂/±25°₂/±88°₂/±21°₂/±14°₄/±88°₂/±11°₄/±88°₄/±18°₂/±88°₂]; the maximum stress is 2074 MPa, and the maximum deformation is 1.128 mm.

In this embodiment, a global optimal solution occurs in a population of the 15th generation, the optimal individual fitness function value is 1.14×10⁻³, and the layer-stacking sequence scheme represented the by optimal individual is [±18°₂/±21°₂/±88°₄/±14°₂/±88°₄/±18°₂/±88°₆/±11°₄/±88°₄/±14°₂/±25°₂]; the maximum stress is 1138 MPa, and the maximum deformation is 0.7734 mm.

After the optimization in this embodiment, the maximum filament-direction stress under a minimum burst pressure is reduced by 45%, and the maximum thermal curing deformation is reduced by 31%.

1. A method for optimizing a layer-stacking sequence of a composite material pressure vessel, comprising:
designing of a to-be-optimized composite material pressure vessel based on design objectives and design variables thereof, the design objectives comprising a liner size and a working pressure of the composite material pressure vessel, and the design variables comprising filament winding angles, winding layer thicknesses, and winding layer quantities;
building of a finite element model based on layer-stacking information and boundary conditions of the to-be-optimized composite material pressure vessel, the layer-stacking information being the design variables;
modeling a liner, based on the liner size, of the pressure vessel by solid elements, and modeling composite material layers of the pressure vessel by continuum shell elements;
obtaining required optimization objectives based on the finite element model; and
establishing an optimization model based on the optimization objectives, and obtaining, through iterative optimization, an optimal layer-stacking sequence of the composite material pressure vessel in a case that the design objectives and the design variables are determined, wherein the pressure vessel comprises a body section and a cap section in structure, composite material layers of the body section comprise spirally winding layers and circumferentially winding layers, and composite material layers of the cap section comprise spirally winding layers, and a winding layer quantity of the body section is a winding layer quantity of the cap section, and
a maximum filament-direction stress under a minimum burse pressure is reduced by 45%, and a maximum thermal curing deformation is reduced by 31%.

2. The method for optimizing a layer-stacking sequence of a composite material pressure vessel of claim 1, wherein in step S1,
the filament winding angles are determined by reaming winding;
the filament winding angles of the body section are calculated by the following formula:
$${α}_{i} = {sin}^{- 1} \frac{{r}_{0} + {r}_{i}}{R} ,$$
in the formula, r₀ is a polar hole radius, rᵢ is a reaming radius, R is a liner radius, and αᵢ is a filament winding angle of the body section corresponding to a distinct reaming radius;
the filament winding angles of the cap section are calculated by the following formula:
$${α}_{i} ( r ) = {sin}^{- 1} \frac{{r}_{0} + {r}_{i}}{r} ,$$
in the formula, r is a parallel circle radius at each position of the cap, and αᵢ(r) is a filament winding angle at each position of the cap corresponding to a distinct reaming radius;
the winding layer thicknesses of the body section are calculated by the following formula:
$$\{ \begin{matrix}{t}_{f α} = \frac{{RP}_{b}}{2 K {σ}_{max} {cos}^{2} α} \\ {t}_{f θ} = \frac{{RP}_{b}}{2 {σ}_{max}} ( 2 - {tan}^{2} α )\end{matrix}$$
in the formula, t_{fα.} is a spirally winding layer thickness of the body section, t_{fθ} is a circumferentially winding layer thickness of the body section, P_{b} is a design burst pressure of the body section, K is a filament strength utilization coefficient, σₘₐₓ is a filament tensile strength, and α is the filament winding angle of the body section;
the winding layer thicknesses of the cap section are calculated by the following formula:
$${t}_{f} ( r ) = {\sum }_{i} \sqrt{\frac{{R}^{2} - {( {r}_{0} + {r}_{i} )}^{2}}{{r}^{2} - {( {r}_{0} + {r}_{i} )}^{2}}} {t}_{f α i}$$
in the formula, t_{f}(r) is a winding layer thickness at a distinct parallel circle radius of the cap section, R is the liner radius, r₀ is the polar hole radius, rᵢ is the reaming radius, r is the parallel circle radius at each position of the cap, and t_{fαi} is a winding layer thickness at a distinct filament winding angle of the body section; and
the winding layer quantities are calculated by the following formula:
$$\{ \begin{matrix}M = \frac{{t}_{f α}}{t} \\ N = \frac{{t}_{f θ}}{t}\end{matrix}$$
in the formula, M is the spirally winding layer quantity, N is the circumferentially winding layer quantity, and t is a thickness of a single filament layer.

3. The method for optimizing a layer-stacking sequence of a composite material pressure vessel of claim 2, wherein in step S2, the finite element model of the composite material pressure vessel refers to establishing a static model and a thermodynamic model separately using the same layer-stacking information;
boundary conditions for the static model comprise: a predefined field comprising four analysis steps, which are self-tightening, unloading, working, and bursting in sequence; and
boundary conditions for the thermodynamic model comprise: a predefined field comprising two analysis steps, which are heat preservation and temperature reduction in sequence.

4. The method for optimizing a layer-stacking sequence of a composite material pressure vessel of claim 2, wherein in step S2, the cap section is divided into a plurality of circular rings, and winding layer angles of the cap section are assigned based on different winding layer angles corresponding to different parallel circle radii; a discrete coordinate system is used for setting winding angles for both the cap section and the body section; and a symmetrical winding layer-stacking method is used for layer-stacking.

5. The method for optimizing a layer-stacking sequence of a composite material pressure vessel of claim 1, wherein in step S3, the optimization objectives comprise a minimized maximum filament-direction stress under the minimum burst pressure and a minimized maximum thermal curing deformation.

6. The method for optimizing a layer-stacking sequence of a composite material pressure vessel of claim 1, wherein the generating a new integer code sequence population through a selection operation, a crossover operation, and a mutation operation in step S44 specifically comprises:
in the selection operation: adopting a random ergodic sampling selection, the principle of the random ergodic sampling selection being as follows: partitioning a wheel based on proportions of individual fitness function values in an accumulated population fitness function value, uniformly arranging pointers in a quantity which is a quantity of individuals to be selected, rotating the wheel, and completing selection based on partitions pointed by the pointers;
in the crossover operation: adopting order crossover which specifically comprises: firstly performing adjacent pairing on a population selected by the selection operation, and determining whether the crossover operation occurs based on a crossover probability, and in a case that the crossover operation occurs, randomly selecting two crossover points from chromosomes of two parents P1 and P2; extracting genes between the two points and placing the genes at the same positions of chromosomes of two offsprings O1 and O2; for the offspring O1, arranging the chromosome of the parent P2 in order and deleting genes already existing in the offspring O1 so as to obtain chromosome sequences inherited from the parent P2 of the offspring O1, and successively inserting the chromosome sequences inherited from the parent P2 into vacant positions of the chromosome of the offspring O1 to obtain the offspring O1; obtaining the offspring O2 in the same way; and completing the crossover operation;
in the mutation operation: determining whether the mutation operation occurs based on a mutation probability, and in a case that the mutation operation occurs, randomly selecting two gene codes of individuals for exchange and completing the mutation operation; and
generating a new integer code sequence population through the three operations sequentially, and recombining a temporary population generated by selection, crossover, and mutation operations on a population of the n^{th} generation with part of excellent individuals of the population of the n^{th} generation so as to generate a population of the (n+1)^{th} generation.

7. The method for optimizing a layer-stacking sequence of a composite material pressure vessel according to claim 1, further comprising:
determining an initial population, an iteration quantity, and a fitness function of a layer sequence, the fitness function using the maximum filament-direction stress x under the minimum burst pressure and the maximum thermal curing deformation y of each of individuals in the population as variables, the fitness function being denoted as
$$F ( x , y ) = \frac{1}{xy} ,$$
and a greater fitness function value indicating that an individual is more excellent and has a greater probability to be selected in a selection operation.

8. The method for optimizing a layer-stacking sequence of a composite material pressure vessel according to claim 7 comprising adopting integer coding for the layer sequence, with the layers occurring in pairs at positive and negative angles, each pair of layers being coded with a distinct integer so as to randomly generate an integer code sequence population.

9. The method for optimizing a layer-stacking sequence of a composite material pressure vessel according to claim 8 comprising defining the layer sequence of the composite material pressure vessel by the integer code sequence: [a₁, a₂, . . . , a_{(n÷m)/2}], the integer code sequence having
$$\frac{n + m}{2}$$
integers, with
$$\frac{n}{2}$$
integers representing circumferentially winding layers and
$$\frac{m}{2}$$
integers representing spirally winding layers, n being a circumferentially winding layer quantity and m being a spirally winding layer quantity.

10. The method for optimizing a layer-stacking sequence of a composite material pressure vessel according to claim 9 comprising generating a layer sequence population based on layer-stacking angles represented by the integer codes, with each individual in the population representing a possible layer-stacking mode of the composite material pressure vessel.

11. The method for optimizing a layer-stacking sequence of a composite material pressure vessel according to claim 10 comprising performing calculation on the layer sequence population to obtain the maximum filament-direction stress under the minimum burst pressure and the maximum thermal curing deformation corresponding to each individual in the population, and calculating the fitness function value corresponding to each individual in the population.

12. The method for optimizing a layer-stacking sequence of a composite material pressure vessel according to claim 7 comprising generating a new integer code sequence population through a selection operation, a crossover operation, and a mutation operation, decoding the integer code sequence to generate a layer sequence population, and calculating corresponding fitness function values.

13. The method for optimizing a layer-stacking sequence of a composite material pressure vessel according to claim 12 comprising determining whether the iteration quantity is reached, and in a case that the iteration quantity is reached, outputting an optimal individual, and in a case that the iteration quantity is not reached, performing the generating of the new integer code sequence population, iteratively.
